(19) United States
(12) Patent Application Publication
(10) Pub. No.: US 2004/0030504 A1
(43) Pub. Date: Feb. 12, 2004

(54) System, method, and computer program product for the representation of biological sequence data

(75) Inventors: Gregg A. Helt, Healdsburg, CA (US); Cyrus L. Harmon, Berkeley, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, Inc. a Corporation Organized under the laws of Delaware

(21) Appl. No.: 10/423,530

(22) Filed: Apr. 25, 2003

Related U.S. Application Data
(60) Provisional application No. 60/375,907, filed on Apr. 26, 2002.
(60) Provisional application No. 60/443,983, filed on Jan. 30, 2003.
(60) Provisional application No. 60/444,952, filed on Feb. 3, 2003.

(51) Int. Cl. G06F 19/00; G01N 33/48; G01N 33/50

(52) U.S. Cl. 702/20

(57) ABSTRACT

A method is described that includes the acts of receiving related biological sequences; and representing each of the related biological sequences as objects of a data model, where at least one of the objects is enabled to map at least one of the related biological sequences with at least one other of the related biological sequences.

### RELATED APPLICATIONS

The present application claims priority to U.S. Provisional Patent Application Serial Nos. 60/375,907, titled “Method, System, and Computer Software for Representing Relationships Between Biological Sequences”, filed Apr. 26, 2002; No. 60/443,983, titled “System, Method and Computer Program Product for Dynamic Display and Analysis of Biological Sequence Data”, filed Jan. 30, 2003; and No. 60/444,952, titled “DAS2: A Distributed Genome Annotation System”, filed Feb. 3, 2003, each of which is hereby incorporated herein by reference in its entirety for all purposes. The present application is also related to U.S. Patent Application Attorney Docket No. 3471.2, titled “System, Method, and Computer Program Product for the Dynamic Display and Analysis of Biological Sequence Data”, filed concurrently herewith, which is hereby incorporated by reference herein in its entirety for all purposes.

### FIELD OF THE INVENTION

The present invention relates to the field of bioinformatics. In particular, the present invention relates to systems, methods, and computer program products for dynamically displaying biological sequence information and providing biological sequence analysis tools that utilize a data model to represent biological sequence information.

### BACKGROUND

Research in molecular biology, biochemistry, and many related health fields increasingly requires organization and analysis of complex data generated by new experimental techniques. These tasks are addressed by the rapidly evolving field of bioinformatics. See, e.g., H. Rashidi and K. Buehler, Bioinformatics Basics: Applications in Biological Science and Medicine (CRC Press, London, 2000); Bioinformatics: A Practical Guide to the Analysis of Gene and Proteins (B. F. Ouelette and A. D. Baxevanis, eds., Wiley & Sons, Inc.; 2d ed., 2001), both of which are hereby incorporated herein by reference in their entireties. Broadly, one area of bioinformatics applies computational techniques to large genomic databases, often distributed over and accessed through networks such as the Internet, for the purpose of illuminating relationships among gene structure and/or location, protein function, and metabolic processes.

### SUMMARY OF THE INVENTION

The expanding use of microarray technology is one of the forces driving the development of bioinformatics. In particular, microarrays and associated instrumentation and computer systems have been developed for rapid and large-scale collection of data about the expression of genes or expressed sequence tags (EST's) in tissue samples. The data may be used, among other things, to study genetic characteristics and to detect mutations relevant to genetic and other diseases or conditions. More specifically, the data gained through microarray experiments is valuable to researchers because, among other reasons, many disease states can potentially be characterized by differences in the expression levels of various genes, either through changes in the copy number of the genetic DNA or through changes in levels of transcription (e.g., through control of initiation, provision of RNA precursors, or RNA processing) of particular genes. Thus, for example, researchers use microarrays to answer questions such as: Which genes are expressed in cells of a malignant tumor but not expressed in either healthy tissue or tissue treated according to a particular regime? Which genes or EST's are expressed in particular organs but not in others? Which genes or EST's are expressed in particular species but not in others? How does the environment, drugs, or other factors influence gene expression? Data collection is only an initial step, however, in answering these and other questions. Researchers are increasingly challenged to extract biologically meaningful information from the vast amounts of data generated by microarray technologies, and to design follow-on experiments. A need exists to provide researchers with improved tools and information to perform these tasks.

A method for representing biological sequences is described that includes the acts of receiving related biological sequences; and representing each of the related biological sequences as objects of a data model, where at least one of the objects is enabled to map at least one of the related biological sequences with at least one other of the related biological sequences.

In some implementations, the related biological sequences include, protein, mRNA, genomic, and probe sequences. The objects include a BioSeq object, where the BioSeq object represents the length and sequence composition of a subsequence of one of the plurality of related biological sequences. A SeqSpan object, where the SeqSpan object represents the start and end positions of the subsequence and points to the BioSeq object. A SeqSymmetry object, where the SeqSymmetry object represents and points to one or more of the SeqSpan objects. An AnnotatedBioseq object, where the AnnotatedBioseq object represents and points to the SeqSymmetry object and one or more SeqSpan objects associated with at least one other of the plurality of related biological sequences.

A system for representing biological sequences is described that includes an input manager that receives related biological sequences; and a dynamic display generator that represents each of the related biological sequences as objects of a data model, where at least one of the objects is enabled to map at least one of the related biological sequences with at least one other of the related biological sequences.

The above implementations are not necessarily inclusive or exclusive of each other and may be combined in any manner that is non-conflicting and otherwise possible, whether they be presented in association with a same, or a different, aspect or implementation. The description of one implementation is not intended to be limiting with respect to other implementations. Also, any one or more function, step, operation, or technique described elsewhere in this specification may, in alternative implementations, be combined with any one or more function, step, operation, or technique described in the summary. Thus, the above implementations are illustrative rather than limiting.

### BRIEF DESCRIPTION OF THE DRAWINGS

The above and further advantages will be more clearly appreciated from the following detailed description when taken in conjunction with the accompanying drawings. In the drawings, like reference numerals indicate like structures or method steps and the leftmost one or two digits of a reference numeral indicate the number of the figure in which the referenced element first appears (for example, the element 180 appears first in FIG. 1; element 1110 appears first in FIG. 11). In functional block diagrams, rectangles generally indicate functional elements, parallelograms generally indicate data, rectangles with curved sides generally indicate stored data, rectangles with a pair of double borders generally indicate predefined functional elements, and keystone shapes generally indicate manual operations. In method flow charts, rectangles generally indicate method steps and diamond shapes generally indicate decision elements. All of these conventions, however, are intended to be typical or illustrative, rather than limiting.

FIG. 1 is a functional block diagram of one embodiment of a dynamic display and analysis system including an illustrative user computer system;

FIG. 2 is a functional block diagram of one embodiment of dynamic display applications as illustratively stored for execution in system memory of the computer system of FIG. 1;

FIG. 3 is a functional block diagram of one embodiment of a conventional system for obtaining biological sequence information over the Internet;

FIG. 4 is a simplified graphical representation of one embodiment of a graphical user interface provided by the dynamic display applications of FIG. 1 that includes an example of a plurality of display panes;

FIG. 5 is a simplified graphical representation of one embodiment of a graphical user interface provided by the dynamic display applications of FIG. 1 that includes an example of a file pull down menu;

FIG. 6 is a simplified graphical representation of one embodiment of a graphical user interface provided by the dynamic display applications of FIG. 1 that includes an example of a view pull down menu;

FIG. 7 is a simplified graphical representation of one embodiment of a graphical user interface provided by the dynamic display applications of FIG. 1 that includes an example of a bookmark pull down menu;

FIG. 8 is a simplified graphical representation of one embodiment of a graphical user interface provided by the dynamic display applications of FIG. 1 that includes an example of a user selection;

FIG. 9 is a simplified graphical representation of one embodiment of a graphical user interface provided by the dynamic display applications of FIG. 1 that includes an example of a right click selection menu;

FIG. 10 is a simplified graphical representation of one embodiment of a graphical user interface provided by the dynamic display applications of FIG. 1 that includes an example of a slicing pad adjustment window;

FIG. 11 is a simplified graphical representation of one embodiment of a graphical user interface provided by the dynamic display applications of FIG. 1 that includes an example of an edge sensitivity adjuster;

FIG. 12 is a simplified graphical representation of one embodiment of a graphical user interface provided by the dynamic display applications of FIG. 1 that includes an example of a user selected graph;

FIG. 13 is a simplified graphical representation of one embodiment of a graphical user interface provided by the dynamic display applications of FIG. 1 that includes an example of a primer design tab;

FIG. 14 is a simplified graphical representation of one embodiment of a graphical user interface provided by the dynamic display applications of FIG. 1 that includes an example of a BLAT mapping tab;

FIG. 15 is a simplified graphical representation of one embodiment of a graphical user interface provided by the dynamic display applications of FIG. 1 that includes an example of an ORF tab;

FIG. 16 is a simplified graphical representation of one embodiment of a graphical user interface provided by the dynamic display applications of FIG. 1 that includes an example of a pattern search tab;

FIG. 17 is a simplified graphical representation of one embodiment of a graphical user interface provided by the dynamic display applications of FIG. 1 that includes an example of a restriction site tab;

FIG. 18 is a simplified graphical representation of one embodiment of a graphical user interface provided by the dynamic display applications of FIG. 1 that includes an example of a DAS window and a curation menu; and

FIG. 19 is a simplified graphical representation of one embodiment of a biological sequence data model as utilized by the dynamic display applications of FIG. 2.

### DETAILED DESCRIPTION

The present invention has many preferred embodiments that, in some instances, may include material incorporated from patents, applications and other references for details known to those of the art. When a patent or patent application is referred to below, it should be understood that it is incorporated by reference in its entirety for all purposes.

As used in this application, the singular form “a,” “an,” and “the” include plural references unless the context clearly dictates otherwise. For example, the term “an agent” includes a plurality of agents, including mixtures thereof. An individual is not limited to a human being but may also be other organisms including but not limited to mammals, plants, bacteria, or cells derived from any of the above.

Throughout this disclosure, various aspects of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This principle applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques may be had by reference to the examples herein. However, other equivalent conventional procedures may, of course, also be used. Such conventional techniques and descriptions may be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The practice of the present invention may also employ conventional biology methods, software, and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes, and other known devices or media and those that may be developed in the future. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Baxevanis Bioinformatics: A Practical Guide for Analysis of Gene and Proteins (Wiley & Sons, Inc., 2nd ed., 2001).

As will be appreciated by one of skill in the art, the present invention may be embodied as a method, data processing system or program products. Accordingly, the present invention may take the form of data analysis systems, methods, analysis software, and so on. Software written according to the present invention typically is to be stored in some form of computer readable medium, such as memory, or CD-ROM, or transmitted over a network, and executed by a processor. For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both of which are hereby incorporated by reference for all purposes.

Computer software products may be written in any of various suitable programming languages, such as C, C++, Fortran and Java (Sun Microsystems®). The computer software product may be an independent application with data input and data display modules. Alternatively, the computer software products may be classes that may be instantiated as distributed objects. The computer software products may also be component software such as Java Beans (Sun Microsystems®), Enterprise Java Beans (EJB), Microsoft® COM/DCOM, etc.

Probe Arrays 103: Various techniques and technologies may be used for synthesizing dense arrays of biological materials on or in a substrate or support. For example, Affymetrix® GeneChip® arrays are synthesized in accordance with techniques sometimes referred to as VLSIPS™ (Very Large Scale Immobilized Polymer Synthesis) technologies. Some aspects of VLSIPS™ and other microarray and polymer (including protein) array manufacturing methods and techniques have been described in U.S. Ser. No. 09/536,841, WO 00/58516, U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752, in PCT Applications Nos. PCT/US99/00730 (International Publication Number WO 99/36760) and PCT/US01/04285, which are all incorporated herein by reference in their entireties for all purposes.

Patents that describe synthesis techniques in specific embodiments include U.S. Pat. Nos. 5,412,087, 6,147,205, 6,262,216, 6,310,189, 5,889,165, and 5,959,098, hereby incorporated by reference in their entireties for all purposes. Nucleic acid arrays are described in many of the above patents, but the same techniques may be applied to polypeptide arrays.

Generally speaking, an “array” typically includes a collection of molecules that can be prepared either synthetically or biosynthetically. The molecules in the array may be identical, they may be duplicative, and/or they may be different from each other. The array may assume a variety of formats, e.g., libraries of soluble molecules; libraries of compounds tethered to resin beads, silica chips, or other solid supports; and other formats.

The terms “solid support,” “support,” and “substrate” may in some contexts be used interchangeably and may refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or other separation members or elements. In some embodiments, the solid support(s) may take the form of beads, resins, gels, microspheres, or other materials and/or geometric configurations.

Generally speaking, a “probe” typically is a molecule that can be recognized by a particular target. To ensure proper interpretation of the term “probe” as used herein, it is noted that contradictory conventions exist in the relevant literature. The word “probe” is used in some contexts to refer not to the biological material that is synthesized on a substrate or deposited on a slide, as described above, but to what is referred to herein as the “target.”

A target is a molecule that has an affinity for a given probe. Targets may be naturally-occurring or man-made molecules. Also, they can be employed in their unaltered state or as aggregates with other species. The samples or targets are processed so that, typically, they are spatially associated with certain probes in the probe array. For example, one or more tagged targets may be distributed over the probe array.

Targets may be attached, covalently or noncovalently, to a binding member, either directly or via a specific binding substance. Examples of targets that can be employed in accordance with this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. Typically, a “probe-target pair” is formed when two macromolecules have combined through molecular recognition to form a complex.

The probes of the arrays in some implementations comprise nucleic acids that are synthesized by methods including the steps of activating regions of a substrate and then contacting the substrate with a selected monomer solution. The term “monomer” generally refers to any member of a set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone. In addition, the terms “biopolymer” and “biological polymer” generally refer to repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer. Related to the term “biopolymer” is the term “biomonomer” that generally refers to a single unit of biopolymer, or a single unit that is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

As used herein, nucleic acids may include any polymer or oligomer of nucleosides or nucleotides (polynucleotides or oligonucleotides) that include pyrimidine and/or purine bases, preferably cytosine, thymine, and uracil, and adenine and guanine, respectively. An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide in accordance with the present invention may be peptide nucleic acid (PNA) in which the constituent bases are joined by peptides bonds rather than phosphodiester linkage, as described in Nielsen et al., Science 254:1497-1500 (1991); Nielsen, Curr. Opin. Biotechnol., 10:71-75 (1999), both of which are hereby incorporated by reference herein. The invention also encompasses situations in which there is a nontraditional base pairing such as Hoogsteen base pairing that has been identified in certain tRNA molecules and postulated to exist in a triple helix. “Polynucleotide” and “oligonucleotide” may be used interchangeably in this application.

Additionally, nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

As noted, a nucleic acid library or array typically is an intentionally created collection of nucleic acids that can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids that can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. Nucleic acid arrays that are useful in the present invention include those that are commercially available from Affymetrix, Inc. of Santa Clara, Calif., under the registered trademark “GeneChip®.” Example arrays are shown on the website at affymetrix.com.

In some embodiments, a probe may be surface immobilized. Examples of probes that can be investigated in accordance with this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies. As non-limiting examples, a probe may refer to a nucleic acid, such as an oligonucleotide, capable of binding to a target nucleic acid of complementary sequence through one or more types of chemical bonds, usually through complementary base pairing, usually through hydrogen bond formation. A probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, or any ligands for detecting its binding partners. Probes of other biological materials, such as peptides or polysaccharides as non-limiting examples, may also be formed. For more details regarding possible implementations, see U.S. Pat. No. 6,156,501, hereby incorporated by reference herein in its entirety for all purposes. When referring to targets or probes as nucleic acids, it should be understood that these are illustrative embodiments that are not to limit the invention in any way.

Furthermore, to avoid confusion, the term “probe” is used herein to refer to probes such as those synthesized according to the VLSIPS™ technology; the biological materials deposited so as to create spotted arrays; and materials synthesized, deposited, or positioned to form arrays according to other current or future technologies. Thus, microarrays formed in accordance with any of these technologies may be referred to generally and collectively hereafter for convenience as “probe arrays.” Moreover, the term “probe” is not limited to probes immobilized in array format. Rather, the functions and methods described herein may also be employed with respect to other parallel assay devices. For example, these functions and methods may be applied with respect to probe-set identifiers that identify probes immobilized on or in beads, optical fibers, or other substrates or media.

In accordance with some implementations, some targets hybridize with probes and remain at the probe locations, while non-hybridized targets are washed away. These hybridized targets, with their tags or labels, are thus spatially associated with the probes. The term “hybridization” refers to the process in which two single-stranded polynucleotides bind non-covalently to form a stable double-stranded polynucleotide. The term “hybridization” may also refer to triple-stranded hybridization, which is theoretically possible. The resulting (usually) double-stranded polynucleotide is a “hybrid.” The proportion of the population of polynucleotides that forms stable hybrids is referred to herein as the “degree of hybridization”. Hybridization probes usually are nucleic acids (such as oligonucleotides) capable of binding in a base-specific manner to a complementary strand of nucleic acid. Such probes include peptide nucleic acids, as described in Nielsen et al., Science 254:1497-1500 (1991) or Nielsen Curr. Opin. Biotechnol., 10:71-75 (1999) (both of which are hereby incorporated herein by reference), and other nucleic acid analogs and nucleic acid mimetics. The hybridized probe and target may sometimes be referred to as a probe-target pair. Detection of these pairs can serve a variety of purposes, such as to determine whether a target nucleic acid has a nucleotide sequence identical to or different from a specific reference sequence. See, for example, U.S. Pat. No. 5,837,832, referred to and incorporated above. Other uses include gene expression monitoring and evaluation (see, e.g., U.S. Pat. No. 5,800,992 to Fodor, et al.; U.S. Pat. No. 6,040,138 to Lockhart, et al.; and International App. No. PCT/US98/15151, published as WO99/05323, to Balaban, et al.), genotyping (U.S. Pat. No. 5,856,092 to Dale, et al.), or other detection of nucleic acids. The '992, '138, and '092 patents, and publication WO99/05323, are incorporated by reference herein in their entireties for all purposes.

The present invention also contemplates signal detection of hybridization between probes and targets in certain preferred embodiments. See U.S. Pat. Nos. 5,143,854, 5,578,832; 5,631,734; 5,936,324; 5,981,956; 6,025,601 incorporated above and in U.S. Pat. Nos. 5,834,758, 6,141,096; 6,185,030; 6,201,639; 6,218,803; and 6,225,625, in U.S. Patent application No. 60/364,731 and in PCT Application PCT/US99/06097 (published as WO99/47964), each of which also is hereby incorporated by reference in its entirety for all purposes.

A system and method for efficiently synthesizing probe arrays using masks is described in U.S. patent application Ser. No. 09/824,931, filed Apr. 3, 2001, that is hereby incorporated by reference herein in its entirety for all purposes. A system and method for a rapid and flexible microarray manufacturing and online ordering system is described in U.S. Provisional Patent Application Serial No. 60/265,103 filed Jan. 29, 2001, that also is hereby incorporated herein by reference in its entirety for all purposes. Systems and methods for optical photolithography without masks are described in U.S. Pat. No. 6,271,957 and in U.S. patent application Ser. No. 09/683,374 filed Dec. 19, 2001, both of which are hereby incorporated by reference herein in their entireties for all purposes.

As noted, various techniques exist for depositing probes on a substrate or support. For example, “spotted arrays” are commercially fabricated, typically on microscope slides. These arrays consist of liquid spots containing biological material of potentially varying compositions and concentrations. For instance, a spot in the array may include a few strands of short oligonucleotides in a water solution, or it may include a high concentration of long strands of complex proteins. The Affymetrix® 417™ Arrayer and 427™ Arrayer are devices that deposit densely packed arrays of biological materials on microscope slides in accordance with these techniques. Aspects of these and other spot arrayers are described in U.S. Pat. Nos. 6,040,193 and 6,136,269 and in PCT Application No. PCT/US99/00730 (International Publication Number WO 99/36760) incorporated above and in U.S. patent application Ser. No. 09/683,298 hereby incorporated by reference in its entirety for all purposes. Other techniques for generating spotted arrays also exist. For example, U.S. Pat. No. 6,040,193 to Winkler, et al. is directed to processes for dispensing drops to generate spotted arrays. The '193 patent, and U.S. Pat. No. 5,885,837 to Winkler, also describe the use of micro-channels or micro-grooves on a substrate, or on a block placed on a substrate, to synthesize arrays of biological materials. These patents further describe separating reactive regions of a substrate from each other by inert regions and spotting on the reactive regions. The '193 and '837 patents are hereby incorporated by reference in their entireties. Another technique is based on ejecting jets of biological material to form a spotted array. Other implementations of the jetting technique may use devices such as syringes or piezo electric pumps to propel the biological material. It will be understood that the foregoing are non-limiting examples of techniques for synthesizing, depositing, or positioning biological material onto or within a substrate. For example, although a planar array surface is preferred in some implementations of the foregoing, a probe array may be fabricated on a surface of virtually any shape or even a multiplicity of surfaces. Arrays may comprise probes synthesized or deposited on beads, fibers such as fiber optics, glass, silicon, silica or any other appropriate substrate, see U.S. Pat. No. 5,800,992 referred to and incorporated above and U.S. Pat. Nos. 5,770,358, 5,789,162, 5,708,153 and 6,361,947 all of which are hereby incorporated in their entireties for all purposes. Arrays may be packaged in such a manner as to allow for diagnostics or other manipulation in an all inclusive device, see for example, U.S. Pat. Nos. 5,856,174 and 5,922,591 hereby incorporated in their entireties by reference for all purposes.

Probes typically are able to detect the expression of corresponding genes or EST's by detecting the presence or abundance of mRNA transcripts present in the target. This detection may, in turn, be accomplished in some implementations by detecting labeled cRNA that is derived from cDNA derived from the mRNA in the target.

The terms “mRNA” and “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

In general, a group of probes, sometimes referred to as a probe set, contains sub-sequences in unique regions of the transcripts and does not correspond to a full gene sequence. Further details regarding the design and use of probes and probe sets are provided in PCT Application Serial No. PCT/US 01/02316, filed Jan. 24, 2001 incorporated above; and in U.S. Pat. No. 6,188,783 and in U.S. patent application Ser. No. 09/721,042, filed on Nov. 21, 2000, Ser. No. 09/718,295, filed on Nov. 21, 2000, Ser. No. 09/745,965, filed on Dec. 21, 2000, and Ser. No. 09/764,324, filed on Jan. 16, 2001, all of which patent and patent applications are hereby incorporated herein by reference in their entireties for all purposes.

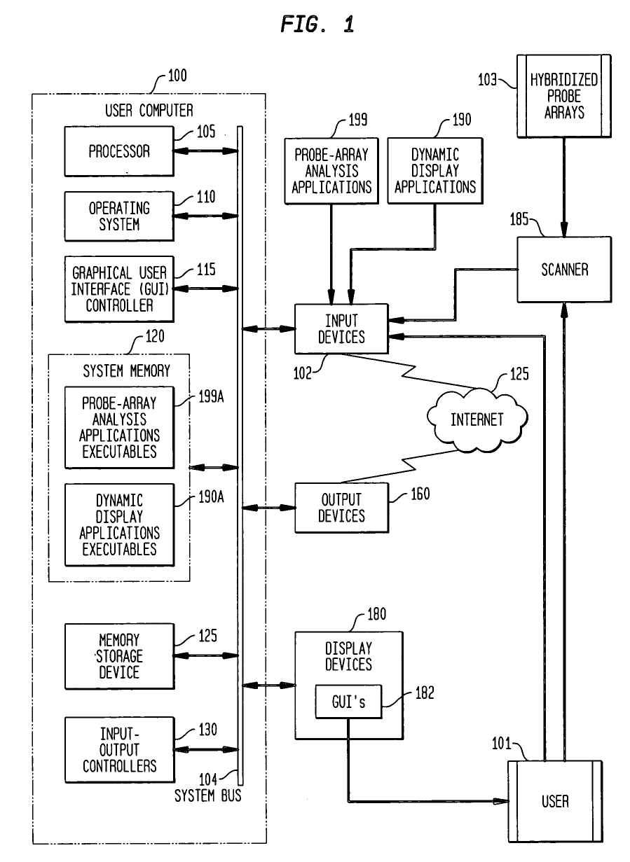

Scanner 190: FIG. 1 is a functional block diagram of a system that is suitable for, among other things, analyzing probe arrays that have been hybridized with labeled targets. Representative hybridized probe arrays 103 of FIG. 1 may include probe arrays of any type, as noted above. Labeled targets in hybridized probe arrays 103 may be detected using various commercial devices, referred to for convenience hereafter as “scanners.” An illustrative device is shown in FIG. 1 as scanner 190. In some implementations, scanners image the targets by detecting fluorescent or other emissions from the labels, or by detecting transmitted, reflected, or scattered radiation. These processes are generally and collectively referred to hereafter for convenience simply as involving the detection of “emissions.” Various detection schemes are employed depending on the type of emissions and other factors. A typical scheme employs optical and other elements to provide excitation light and to selectively collect the emissions. Also included in some implementations are various light-detector systems employing photodiodes, charge-coupled devices, photomultiplier tubes, or similar devices to register the collected emissions.

Methods and apparatus for signal detection and processing of intensity data are disclosed in, for example, U.S. Pat. Nos. 5,143,854, 5,578,832, 5,631,734, 5,800,992, 5,834,758, 5,856,092, 5,936,324, 5,981,956, 6,025,601, 6,090,555, 6,141,096, 6,185,030, 6,201,639, 6,207,960, 6,218,803, 6,225,625, in PCT Application PCT/US99/06097 (published as WO99/47964) incorporated above, and in U.S. Pat. Nos. 5,547,839, 5,902,723, 6,171,793, 6,207,960, 6,252,236, 6,335,824, 6,490,533, 6,472,671, 6,403,320, and 6,407,858 each of which is hereby incorporated by reference in its entirety for all purposes. Other scanners or scanning systems are described in U.S. patent application Ser. No. 09/682,837 filed Oct. 23, 2001; Ser. No. 09/683,216 filed Dec. 3, 2001; Ser. No. 09/683,217 filed Dec. 3, 2001; Ser. No. 09/683,219 filed Dec. 3, 2001; and Ser. No. 10/389,194, filed Mar. 14, 2003, each of which is hereby incorporated by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,974,164, 6,090,555, 6,188,783 incorporated above and U.S. Pat. Nos. 5,733,729, 6,066,454, 6,185,561, 6,223,127, 6,229,911 and 6,308,170, hereby incorporated herein in their entireties for all purposes.

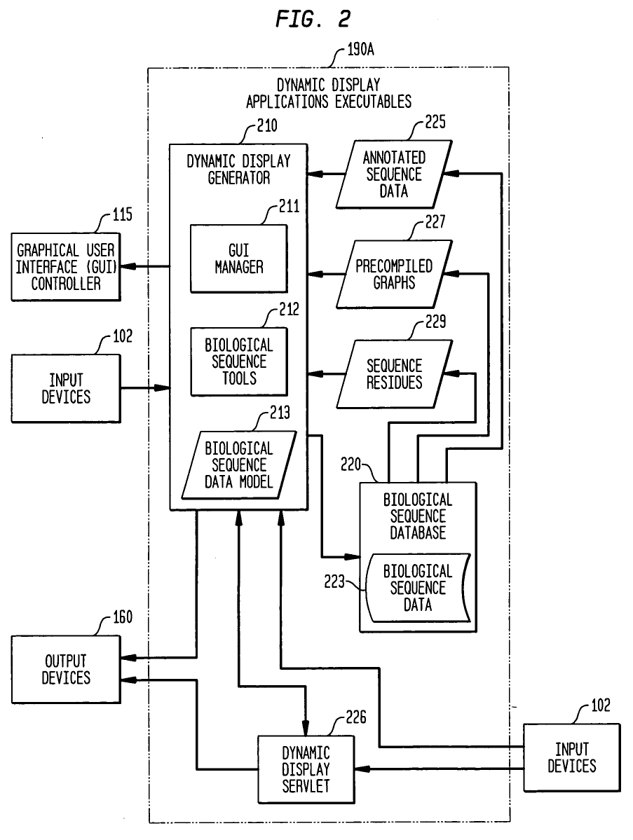

Scanner 185 provides data representing the intensities (and possibly other characteristics, such as color) of the detected emissions, as well as the locations on the substrate where the emissions were detected. The data typically are stored in a memory device, such as system memory 120 of user computer 100, in the form of a data file or other data storage form or format. One type of data file, such as image data file 212 shown in FIG. 2, typically includes intensity and location information corresponding to elemental sub-areas of the scanned substrate. The term “elemental” in this context means that the intensities, and/or other characteristics, of the emissions from this area each are represented by a single value. When displayed as an image for viewing or processing, elemental picture elements, or pixels, often represent this information. Thus, for example, a pixel may have a single value representing the intensity of the elemental sub-area of the substrate from which the emissions were scanned. The pixel may also have another value representing another characteristic, such as color. For instance, a scanned elemental sub-area in which high-intensity emissions were detected may be represented by a pixel having high luminance (hereafter, a “bright” pixel), and low-intensity emissions may be represented by a pixel of low luminance (a “dim” pixel). Alternatively, the chromatic value of a pixel may be made to represent the intensity, color, or other characteristic of the detected emissions. Thus, an area of high-intensity emission may be displayed as a red pixel and an area of low-intensity emission as a blue pixel. As another example, detected emissions of one wavelength at a particular sub-area of the substrate may be represented as a red pixel, and emissions of a second wavelength detected at another sub-area may be represented by an adjacent blue pixel. Many other display schemes are known. Two examples of image data are data files in the form *.dat or *.tif as generated respectively by Affymetrix® Microarray Suite or Affymetrix® GeneChip® Operating Software based on images scanned from GeneChip® arrays, and by Affymetrix® Jaguar™ software based on images scanned from spotted arrays.

Probe-Array Analysis Applications 199: Generally, a human being may inspect a printed or displayed image constructed from the data in an image file and may identify those cells that are bright or dim, or are otherwise identified by a pixel characteristic (such as color). However, it frequently is desirable to provide this information in an automated, quantifiable, and repeatable way that is compatible with various image processing and/or analysis techniques. For example, the information may be provided for processing by a computer application that associates the locations where hybridized targets were detected with known locations where probes of known identities were synthesized or deposited. Other methods include tagging individual synthesis or support substrates (such as beads) using chemical, biological, electromagnetic transducers or transmitters, and other identifiers. Information such as the nucleotide or monomer sequence of target DNA or RNA may then be deduced. Techniques for making these deductions are described, for example, in U.S. Pat. No. 5,733,729 and in U.S. Pat. No. 5,837,832, noted and incorporated above.

A variety of computer software applications are commercially available for controlling scanners (and other instruments related to the hybridization process, such as hybridization chambers), and for acquiring and processing the image files provided by the scanners. Examples are the Jaguar™ application from Affymetrix, Inc., aspects of which are described in PCT Application PCT/US 01/26390, and PCT/US 01/226297, and in U.S. patent application Ser. Nos. 09/681,819, 09/682,071, 09/682,074, and 09/682,076, the Microarray Suite application from Affymetrix, Inc., aspects of which are described in U.S. patent application Ser. Nos. 09/683,912, 10/219,503, 10/219,882, and 10/370,442, and the GeneChip® Operating Software from Affymetrix, Inc., aspects of which are described in U.S. Provisional Patent Application No. 60/442,684, all of which are hereby incorporated herein by reference in their entireties for all purposes. For example, image data in image data file 212 may be operated upon to generate intermediate results such as so-called cell intensity files (*.cel) and chip files (*.chp), generated by Microarray Suite or GeneChip® Operating Software or spot files (*.spt) generated by Jaguar™ software. For convenience, the terms “file” or “data structure” may be used herein to refer to the organization of data, or the data itself generated or used by executables 199A and executable counterparts of other applications. However, it will be understood that any of a variety of alternative techniques known in the relevant art for storing, conveying, and/or manipulating data may be employed, and that the terms “file” and “data structure” therefore are to be interpreted broadly. In the illustrative case in which image data file 212 is derived from a GeneChip® probe array, and in which Microarray Suite or GeneChip® Operating Software generates cell intensity file 216, file 216 may contain, for each probe scanned by scanner 190, a single value representative of the intensities of pixels measured by scanner 185 for that probe. Thus, this value is a measure of the abundance of tagged cRNA's present in the target that hybridized to the corresponding probe. Many such cRNA's may be present in each probe, as a probe on a GeneChip® probe array may include, for example, millions of oligonucleotides designed to detect the cRNA's. The resulting data stored in the chip file may include degrees of hybridization, absolute and/or differential (over two or more experiments) expression, genotype comparisons, detection of polymorphisms and mutations, and other analytical results. In another example, in which executables 199A includes image data from a spotted probe array, the resulting spot file includes the intensities of labeled targets that hybridized to probes in the array. Further details regarding cell files, chip files, and spot files are provided in U.S. patent application Ser. Nos. 09/683,912, 10/219,503, 10/219,882, and 10/370,442, incorporated by reference above.

In the present example, in which executables 199A may include aspects of Affymetrix® Microarray Suite or GeneChip® Operating Software, the chip file is derived from analysis of the cell file combined in some cases with information derived from library files (not shown) that specify details regarding the sequences and locations of probes and controls. Laboratory or experimental data may also be provided to the software for inclusion in the chip file. For example, an experimenter and/or automated data input devices or programs (not shown) may provide data related to the design or conduct of experiments. As a non-limiting example related to the processing of an Affymetrix® GeneChip® probe array, the experimenter may specify an Affymetrix catalog or custom chip type (e.g., Human Genome U95Av2 chip) either by selecting from a predetermined list presented by Microarray Suite or GeneChip® Operating Software or by scanning a bar code related to a chip to read its type. Microarray Suite or GeneChip® Operating Software may associate the chip type with various scanning parameters stored in data tables including the area of the chip that is to be scanned, the location of chrome borders on the chip used for auto-focusing, the wavelength or intensity of laser light to be used in reading the chip, and so on. Other experimental or laboratory data may include, for example, the name of the experimenter, the dates on which various experiments were conducted, the equipment used, the types of fluorescent dyes used as labels, protocols followed, and numerous other attributes of experiments. As noted, executables 199A may apply some of this data in the generation of intermediate results. For example, information about the dyes may be incorporated into determinations of relative expression. Other data, such as the name of the experimenter, may be processed by executables 199A or may simply be preserved and stored in files or other data structures. Any of these data may be provided, for example over a network, to a laboratory information management server computer configured to manage information from large numbers of experiments. Executables 199A may also generate various types of plots, graphs, tables, and other tabular and/or graphical representations of analytical data. As will be appreciated by those skilled in the relevant art, the preceding and following descriptions of files generated by executables 199A are exemplary only, and the data described, and other data, may be processed, combined, arranged, and/or presented in many other ways.

The processed image files produced by these applications often are further processed to extract additional data. In particular, data-mining software applications often are used for supplemental identification and analysis of biologically interesting patterns or degrees of hybridization of probe sets. An example of a software application of this type is the Affymetrix® Data Mining Tool and described in U.S. patent application Ser. No. 09/683,980 which is hereby incorporated herein by reference in its entirety for all purposes. Software applications also are available for storing and managing the enormous amounts of data that often are generated by probe-array experiments and by the image-processing and data-mining software noted above. An example of these data-management software applications is the Affymetrix® Laboratory Information Management System (LIMS) that is described in U.S. patent application Ser. No. 09/682,098 which is hereby incorporated by reference herein in its entirety for all purposes. In addition, various proprietary databases accessed by database management software, such as the Affymetrix® EASI (Expression Analysis Sequence Information) database and database software, provide researchers with associations between probe sets and gene or EST identifiers.

For convenience of reference, these types of computer software applications (i.e., for acquiring and processing image files, data mining, data management, and various database and other applications related to probe-array analysis) are generally and collectively represented in FIG. 1 as probe-array analysis applications 199.

As will be appreciated by those skilled in the relevant art, it is not necessary that applications 199 be stored on and/or executed from computer 100; rather, some or all of applications 199 may be stored on and/or executed from an applications server or other computer platform to which computer 100 is connected in a network. For example, it may be particularly advantageous for applications involving the manipulation of large databases, such as Affymetrix® LIMS or Affymetrix® Data Mining Tool (DMT), to be executed from a database server. Alternatively, LIMS, DMT, and/or other applications may be executed from computer 100, but some or all of the databases upon which those applications operate may be stored for common access on the database server (perhaps together with a database management program, such as the Oracle® 8.0.5 database management system from Oracle Corporation). Such networked arrangements may be implemented in accordance with known techniques using commercially available hardware and software, such as those available for implementing a local-area network or wide-area network.

In some implementations, it may be convenient for user 101 to group probe-set identifiers for batch transfer of information or to otherwise analyze or process groups of probe sets together. For example, as described below, user 101 may wish to obtain annotation information via a portal related to one or more probe sets identified by their respective probe set identifiers. Rather than obtaining this information serially, user 101 may group probe sets together for batch processing. Various known techniques may be employed for associating probe set identifiers, or data related to those identifiers, together. For instance, user 101 may generate a tab delimited *.txt file including a list of probe set identifiers for batch processing. This file or another file or data structure for providing a batch of data (hereafter referred to for convenience simply as a “batch file”), may be any kind of list, text, data structure, or other collection of data in any format. The batch file may also specify what kind of information user 101 wishes to obtain with respect to all, or any combination of, the identified probe sets. In some implementations, user 101 may specify a name or other user-specified identifier to represent the group of probe-set identifiers specified in the text file or otherwise specified by user 101. This user-specified identifier may be stored by one of executables 199A, or by elements of portal 400 described below, so that user 101 may employ it in future operations rather than providing the associated probe-set identifiers in a text file or other format. Thus, for example, user 101 may formulate one or more queries associated with a particular user-specified identifier, resulting in a batch transfer of information from portal 400 to user 101 related to the probe-set identifiers that user 101 has associated with the user-specified identifier. Alternatively, user 101 may initiate a batch transfer by providing the text file of probe-set identifiers. In any of these cases, user 101 may formulate queries to obtain, in a single batch operation, probe set records, lists of probe sets sorted into functional groups, protein domain information, sequence homology information, metabolic pathway information, BLAST similarity searches, array content information, and any other information available via portal 400. Similarly, user 101 may provide information, such as laboratory or experimental information, related to a number of probe sets by a batch operation rather than serial ones. The probe sets may be grouped by experiments, by similarity of probe sets (e.g., probe sets representing genes having similar annotations, such as related to transcription regulation), or any other type of grouping. For example, user 101 may assign a user-specified identifier (e.g., “experiments of January 1”) to a series of experiments and submit probe-set identifiers in user-selected categories (e.g., identifying probe sets that were up-regulated by a specified amount) and provide the experimental information to the portal for data storage and/or analysis.

User Computer 100: User computer 100, shown in FIG. 1, may be a computing device specially designed and configured to support and execute some or all of the functions of probe array applications 199. Computer 100 also may be any of a variety of types of general-purpose computers such as a personal computer, network server, workstation, or other computer platform now or later developed. Computer 100 typically includes known components such as a processor 105, an operating system 110, a graphical user interface (GUI) controller 115, a system memory 120, memory storage devices 125, and input-output controllers 130. It will be understood by those skilled in the relevant art that there are many possible configurations of the components of computer 100 and that some components that may typically be included in computer 100 are not shown, such as cache memory, a data backup unit, and many other devices. Processor 105 may be a commercially available processor such as a Pentium® processor made by Intel Corporation, a SPARC® processor made by Sun Microsystems®, or it may be one of other processors that are or will become available. Processor 105 executes operating system 110, which may be, for example, a Windows®-type operating system (such as Windows NT® 4.0 with SP6a) from the Microsoft Corporation; a Unix® or Linux-type operating system available from many vendors; another or a future operating system; or some combination thereof. Operating system 110 interfaces with firmware and hardware in a well-known manner, and facilitates processor 105 in coordinating and executing the functions of various computer programs that may be written in a variety of programming languages. Operating system 110, typically in cooperation with processor 105, coordinates and executes functions of the other components of computer 100. Operating system 110 also provides scheduling, input-output control, file and data management, memory management, and communication control and related services, all in accordance with known techniques.

System memory 120 may be any of a variety of known or future memory storage devices. Examples include any commonly available random access memory (RAM), magnetic medium such as a resident hard disk or tape, an optical medium such as a read and write compact disc, or other memory storage device. Memory storage device 125 may be any of a variety of known or future devices, including a compact disk drive, a tape drive, a removable hard disk drive, or a diskette drive. Such types of memory storage device 125 typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 120 and/or the program storage device used in conjunction with memory storage device 125.

In some embodiments, a computer program product is described comprising a computer usable medium having control logic (computer software program, including program code) stored therein. The control logic, when executed by processor 105, causes processor 105 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 130 could include any of a variety of known devices for accepting and processing information from a user, whether a human or a machine, whether local or remote. Such devices include, for example, modem cards, network interface cards, sound cards, or other types of controllers for any of a variety of known input devices 102. Output controllers of input-output controllers 130 could include controllers for any of a variety of known display devices 180 for presenting information to a user, whether a human or a machine, whether local or remote. If one of display devices 180 provides visual information, this information typically may be logically and/or physically organized as an array of picture elements, sometimes referred to as pixels. Graphical user interface (GUI) controller 115 may comprise any of a variety of known or future software programs for providing graphical input and output interfaces between computer 100 and user 101, and for processing user inputs. In the illustrated embodiment, the functional elements of computer 100 communicate with each other via system bus 104. Some of these communications may be accomplished in alternative embodiments using network or other types of remote communications.

As will be evident to those skilled in the relevant art, applications 199, if implemented in software, may be loaded into system memory 120 and/or memory storage device 125 through one of input devices 102. All or portions of applications 199 may also reside in a read-only memory or similar device of memory storage device 125, such devices not requiring that applications 199 first be loaded through input devices 102. It will be understood by those skilled in the relevant art that applications 199, or portions of it, may be loaded by processor 105 in a known manner into system memory 120, or cache memory (not shown), or both, as advantageous for execution.

Biological Sequence Data Model 213: Many attempts have been made to represent biological sequence information and the relationships between biological sequences in a machine readable format. For instance the representation may include a data model that focuses on genomic, mRNA, EST, or other type of biological sequence information as well as annotation information associated with the biological sequence information. An illustrative example of a data model is presented in FIG. 2 as data model 213 associated with dynamic display analysis generator 210 that will be described in detail below. The term “data model”, as used herein, generally refers to a representation of one or more elements within a selected type of data that, for instance, may be implemented by a computer database to catalog and store data in a useable fashion. As those of ordinary skill in the related art will appreciate, the data model may include what is referred to as a hierarchical, network, object oriented, object-relational, entity-relationship, or other type of data model. Additionally, data model 213 may be represented using the Unified Modeling Language (commonly referred to as UML), Data Manipulation Language (commonly referred to as DML), or other type of language known to those of ordinary skill in the related art. Some implementations of data model 213 may also utilize BioPerl, Biojava, BioPython, or other types of tools or modules known to those of ordinary skill in the related art.

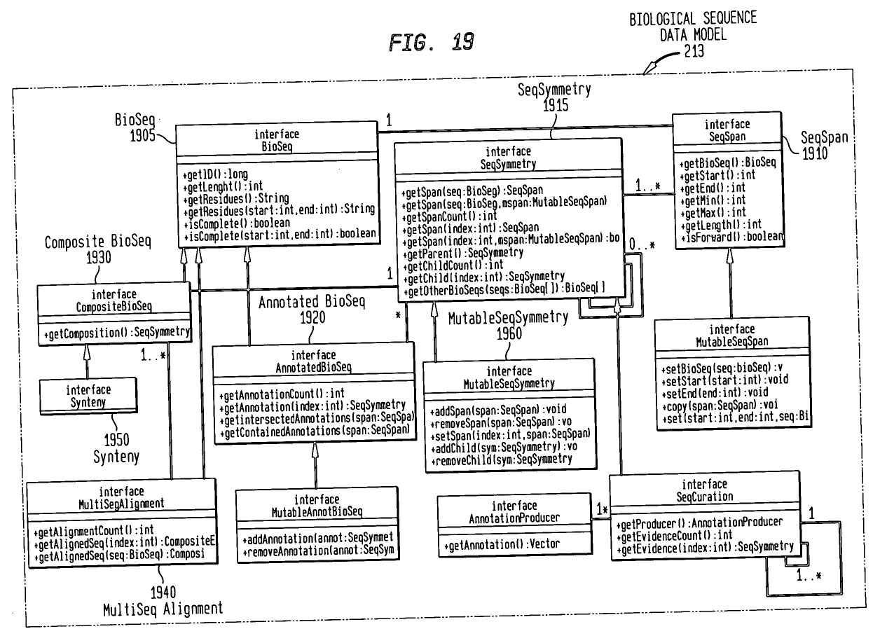

The example of data model 213 is further illustrated in FIG. 19 that includes that illustrates a generalized and unified data model for representing biological sequence and their relationships that may be implemented in what is known to those in the art as an object oriented design philosophy. Annotations are included in what are commonly referred to as objects of the data model as compared, for example, to conventional schemes in which annotations may be associated with sequence information. Also, model 213 may be said to be less hierarchical than traditional annotation methods. For example, a traditional method may use a gene sequence to point to a transcript sequence that in turn points to a protein sequence and subsequently points to the annotation. In contrast, some implementations of model 213 may incorporate annotations directly in the data objects so that the annotation for a gene sequence is found in one or more data objects representing a chromosome, contiguous fragment or sequence, bacterial artificial chromosome, or other genomic sequence entity. In the present example, model 213 offers the user flexibility to manipulate biological sequence information for particular needs and is efficient in both memory and computational time.

As will be appreciated by those skilled in the relevant art, it is not necessary that model 213 be stored on and/or executed from computer 100; rather, some or all of model 213 may be stored on and/or executed from an applications server or other computer platform to which computer 100 is connected in a network. Such networked arrangements may be implemented in accordance with known techniques using commercially available hardware and software, such as those available for implementing a local-area network or wide-area network.

The core data model may include a variety of data objects, such as BioSeq 1905, SeqSpan 1910, and SeqSymmetry 1915. For example, BioSeq 1905 may represent the length of a particular sequence that may, for instance, be a subsequence of a large sequence such as a chromosome, and optionally the residue composition of that sequence or subsequence. SeqSpan 1910 may represent the start point (using a determined point as a reference) of a sequence such as the sequence represented by BioSeq 1905, the end point of the sequence and may further include what is commonly referred to as a pointer to BioSeq 1905. SeqSymmetry 1915 may represent one or more SeqSpan 1910 objects. Thus, in the present example, each SeqSpan 1910 points to a BioSeq 1905 object and each SeqSymmetry 1915 points to one or more SeqSpan 1910 objects.

Additionally, other elements of model 213 may include AnnotatedBioSeq 1920 that may represent a collection of SeqSpan 1910 objects that, for instance, may provide one or more annotations to one or more other sequences associated with the sequence represented by SeqSymmetry 1915 and/or BioSeq 1905. For example, the arrangement of objects in biological sequence data model 213 may offer convenience to a user in that annotations to one or more other related sequences do not have to be independently tracked. Therefore the interfaces or applications utilizing data model 213 may retrieve annotations covered by the span within the sequence. In the present example, networks of annotations may be traversed by alternating between AnnotatedBioSeq 1920 objects and SeqSymmetry 1915 objects.

In some implementations, data model 213 may include a representation of the sequence composition (i.e. the identity of each base or residue within the sequence) illustrated in FIG. 19 as CompositeBioSeq 1930. Each CompositeBioSeq 1930 may include at least one SeqSymmetry 1915 object that represents the mapping of one or more BioSeq 1905 objects used in the composition to the CompositeBioSeq itself. For example, a representation of the sequence composition may be useful for methods known to those in the relevant art as sequence assembly such as assembly of genomic information, or building vectors.

Other possible examples of the utility of a CompositeBioSeq 1930 object may include representing the sequence of an entire chromosome. The chromosome sequence may be subdivided into smaller sequence segments based upon various criteria such as, for instance, intron/exon boundaries that may be more amenable to analysis where sequence segment may be individually represented in the CompositeBioSeq 1930 object. Yet another example may include representing genotypes such as those that have different sequence composition commonly referred to as Single Nucleotide Polymorphisms (SNPs). Still other examples may include what is referred to by those of ordinary skill in the art as primer construction (composing a sequence), reverse complement (returning the reverse of a particular sequence), and coordinate shifting (operations based on reference points).

Some implementations of data model 213 may include a representation of what those of ordinary skill in the related art refer to as multiple sequence alignments, illustrated in FIG. 19 as MultiSeqAlignment 1940. The term “multiple sequence alignment”, generally refers to an alignment of at least two sequences to each other using a variety of available methods that align similar bases in similar locations along the sequence. For example, alignments of multiple sequences may be represented by subdividing the multiple sequence alignment such as, for instance, horizontally where each row (i.e. each sequence aligned) in the alignment is subdivided out. Each subdivided row may be represented as a CompositeBioSeq 1930 object whose composition maps a BioSeq 1905 object of another row to the same coordinate space as the alignment therefore providing a reference to the alignment. In the present example, each row or sequence may be annotated with another row or sequence using the BioSeq 1905 object.

In the same or alternative embodiment, the alignment may additionally be subdivided vertically that may, for instance, provide a reference to the positional relationship of one or more subsequences of one or more bases between the sequences aligned. The vertical subdivisions may, in some implementations provide a representation of what is referred toby those of ordinary skill in the related art as a syntenic relationship. As illustrated in FIG. 19, data model 213 may represent a syntenic as synteny 1950. The term “synteny” commonly refers to the relative positional arrangement of a common sub-sequence of one or more bases between at least two sequences. For example, a four base subsequence “GATT” is common to two related sequences, where the related sequences are said to have a high degree of synteny if the four base subsequence is located at the same position along each related sequence. Conversely the sequences would be referred to as having a low degree of synteny with respect to the subsequences if the arrangement of each subsequence on each related sequence were positioned differently relative to each other.

In some embodiments, the data model may also represent what is referred to as transformations. The term “transformations” as used herein generally refers to methods of mapping a sequence to one or more other sequences. The transformation may include one or more references from one or more sequences to one or more other sequences. For example, a protein sequence, represented as a SeqSymmetry 1915 object, may be transformed by, for instance, using an Annotated BioSeq 1920 object to relate the protein sequence to an associated mRNA sequence that may for instance also be represented as a SeqSymmetry 1915 object. In the present example, the protein and/or mRNA sequences may be represented as a SeqSymmetry 1915 object and/or a MutableSeqSymmetry 1960 object. Similarly, the mRNA sequence may be transformed to a genomic sequence.

Examples of some of possible applications of transformation may include mapping contig annotations to larger genomic assemblies, mapping protein annotations to the genome, mapping genomic annotations to proteins and transcripts, exon structure annotations, and propagation of annotations from one mapping to another.

An additional example of a data model for use with biological sequence information is provided in U.S. Provisional Patent Application Serial No. 60/375,907, titled “Method, System, and Computer Software for Representing Relationships Between Biological Sequences”, filed Apr. 26, 2002, incorporated by reference above.

Dynamic Display Generator 210: In many situations, it may be advantageous for a user have a tool at their disposal that enables the user to visualize and manipulate biological sequence data and related annotation information in a dynamic manner. Such a tool may allow a user to uncover elements hidden within experimental data, such as for example what may be referred to as transcriptome data, alternative splice data, or genotyping data generated from experiments with biological probe arrays. An illustrative example of such a tool is presented in FIG. 2 as generator 210. In the illustrative example generator 210 may be an element of dynamic display applications as shown in FIG. 1, and its executable counterpart dynamic display applications executables 190A. Dynamic display applications executables 190A may comprise a variety of elements including dynamic display analysis generator 210, Local database application biological sequence data 220 that may, for instance, include biological sequence data 223 that represents biological sequence information using biological sequence data model 213, and dynamic display servlet 226.

In some implementations local database 220 may be located on the same workstation as generator 210, although database 220 could be located remotely for instance on a separate workstation or server. Those of ordinary skill in the related art will appreciate that local database 220 may include a relational or other type of database as well as what are commonly referred to as file based database systems. In some implementations, biological sequence data 223 may include annotated sequence data 225, precompiled graphs 227, sequence residues 229, sequence alignment data, sequence search results, or other type of biological sequence related data.

GUI manager 211 of dynamic display analysis generator 210 may provide a graphical user interface that may include a variety of display features and tools provided by biological sequence tools 212. In some implementations GUI manager 211 generates and supports an interactive graphical user interface (hereafter referred to as a GUI, such as GUI 182) that displays biological sequence and related data and is responsive to user selections. Functional elements of generator 210 and other software applications referred to herein, may be implemented using Java or any of a variety of other programming languages. For example, applications may also be written in Microsoft Visual C++, C++, Visual Basic, any other high-level or low-level programming language, or any combination thereof. Also some implementations may include generator 210 that utilizes data model 213 for representing, organizing, and analyzing biological sequence data. Generator 210 receives biological sequence data from a user or some other source via input devices 102, and converts it to biological sequence data 223 using data model 213 to represent the biological sequence data.

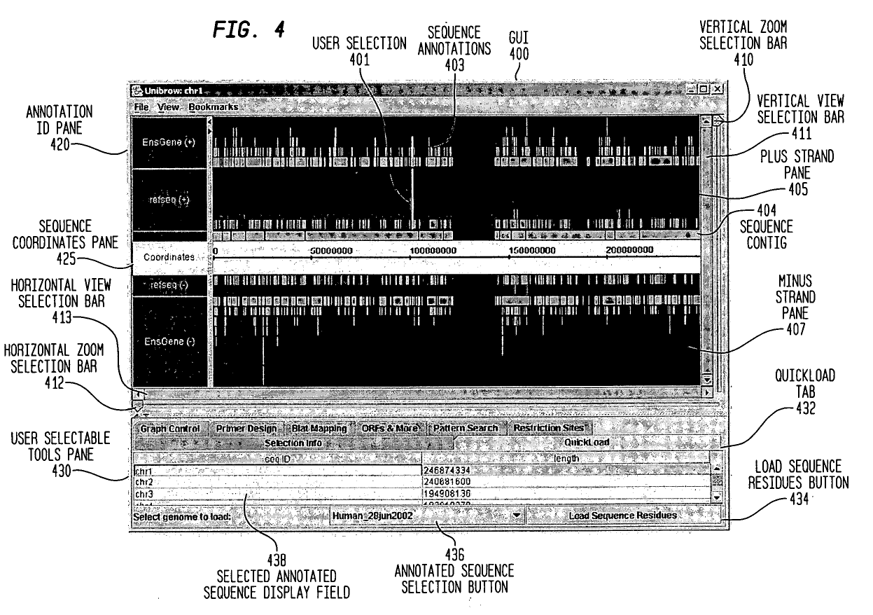

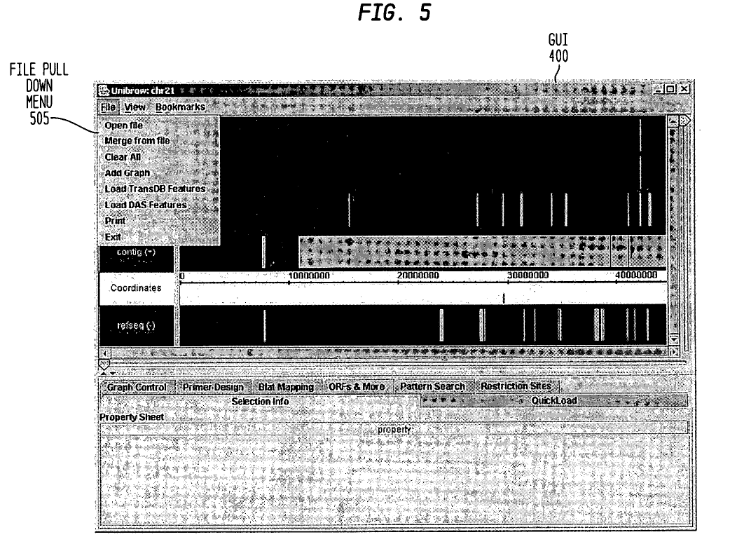

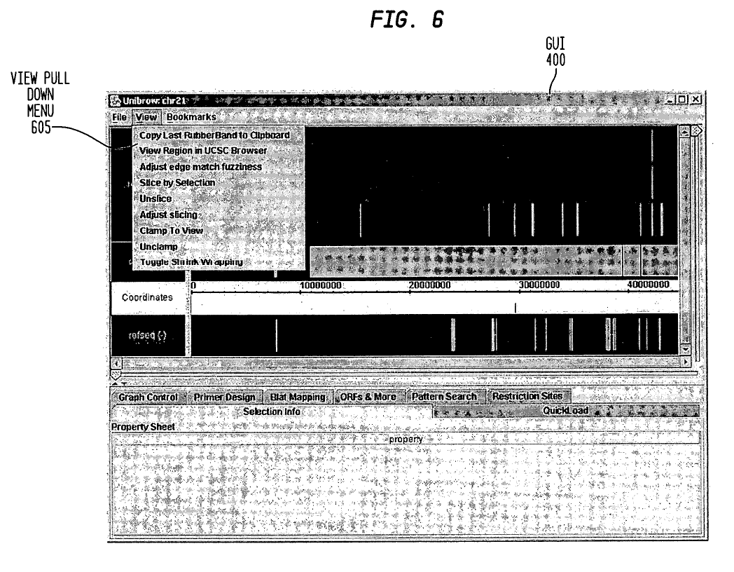

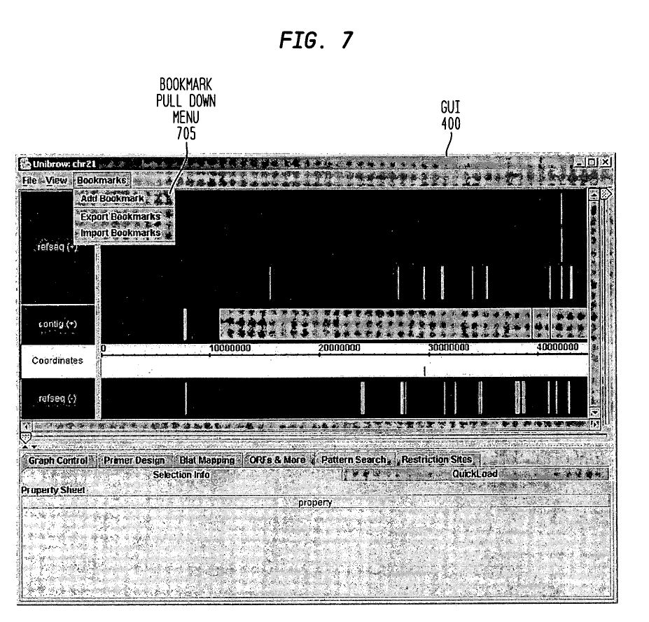

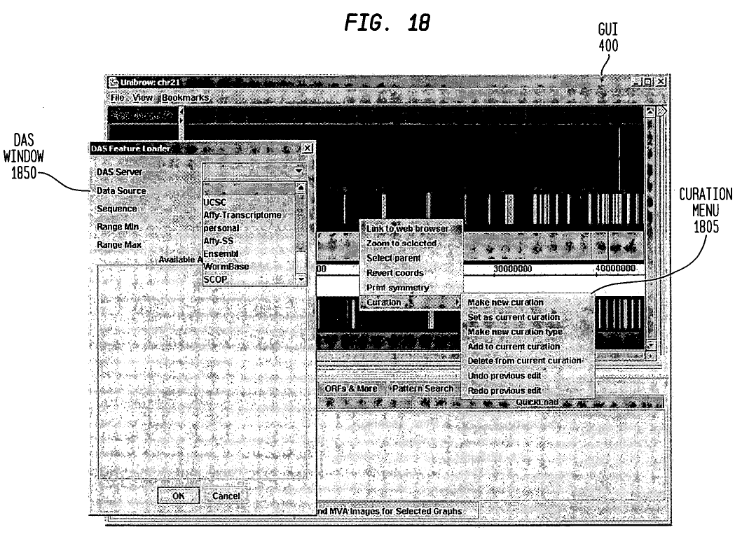

Illustrated in FIGS. 4 through 18 is an example of one possible embodiment of an interactive GUI generated by GUI manager 211. In the illustrative example GUI 400 may include a plurality of panes, such as, for instance plus strand pane 405, minus strand pane 407, annotation ID pane 420, sequence coordinates pane 425, and user selectable tools pane 430. Each of the panes represented in GUI 400 may have a particular purpose.

Pane 405 may display sequence and annotation data that corresponds to what is commonly referred to by those of ordinary skill in the related art as the plus strand of DNA that is also sometimes referred to as the coding strand. Similarly, pane 407 may display similar information as pane 405 except that the displayed information may correspond to the minus strand that may also sometimes be referred to as the non-coding strand. The sequence and annotation information could include, for instance, sequence annotations 403 and sequence contig 404. For example annotations 403 may include sequences with some functional significance, such as predicted exon data from sources such as NCBI RefSeq, Ensembl, or other source of biological sequence data. Contig 404 may include raw and/or more complete sequence data from sources such as the Human Genome Project, or other source of public or private sequence information. In some embodiments annotations 403 may be aligned by sequence position information to contig 404 or other loaded sequence. The graphical representation of contig 404 could include a solid colored bar or other type of pattern that may have gaps in the representation that may represent areas where the biological sequence may be unknown or unverified. In some embodiments a user may interactively move the displayed graphical elements between panes 405 and 407 interchangeably by various methods that includes commonly used methods such as selecting and dragging elements to new locations with a mouse.

Annotation ID pane 420 may include specific identifiers to biological sequence, sequence annotations, or other identifiers that corresponds to and specifically identifies data displayed in panes 405 and 407. Additionally sequence coordinates pane 425 may include a graphical representation of a scale of measurement that may correspond to biological sequence lengths and distances in numbers of sequence bases, kilobases, megabases, centimorgans or other scale of measurement commonly used for biological sequence information.

In some implementations, panes 405, 407, and 425 includes dynamic features that a user may use to control the level of magnification, otherwise referred to as the level of “zoom” of the data. The features may include vertical zoom selection bar 410 and horizontal zoom selection bar 412 that a user may interactively select the level of magnification by methods that include selecting and dragging a graphical element, such as a tab, along the selection bar with a mouse. Increasing the level of magnification of selection bar 410 may, for instance, increase the height in the vertical axis of the graphical representations of the data displayed in panes 405 and 407. Alternatively, decreasing the level of magnification may reduce the height. Possible advantages of controlling the magnification of bar 410 include the customization of the representation of the data viewed in panes 405 and 407, such as to include or exclude particular elements from view in panes 405 and 407 or alternatively to enhance or decrease the resolution of elements displayed within panes 405 and 407 that may, for instance, make differences between elements more apparent to user 101. Similarly, selection bar 410, may allow user 101 to interactively select the level of magnification in selection bar 412. For example, at the lowest degree of magnification an entire sequence and annotations loaded into GUI manager 211 may be entirely displayed in panes 405 and 407 where the corresponding level of resolution of the sequence and related annotations is very low relative to the length of the loaded sequence. As a user increases the level of magnification with selection bar 412, the level of resolution of the loaded sequence and related annotation data increases proportional to the position of the graphical element along selection bar 412, and relative to the overall length of the loaded sequence. Similarly, the resolution of the scale displayed in coordinates pane 425 may increase or decrease corresponding to the selected level of magnification of bar 412. In the present example, as the resolution increases the amount of data displayed in panes may be decreased, such that some of the sequence related information “scrolls” off one or both of the vertical and/or horizontal edges of panes 405 and 407.

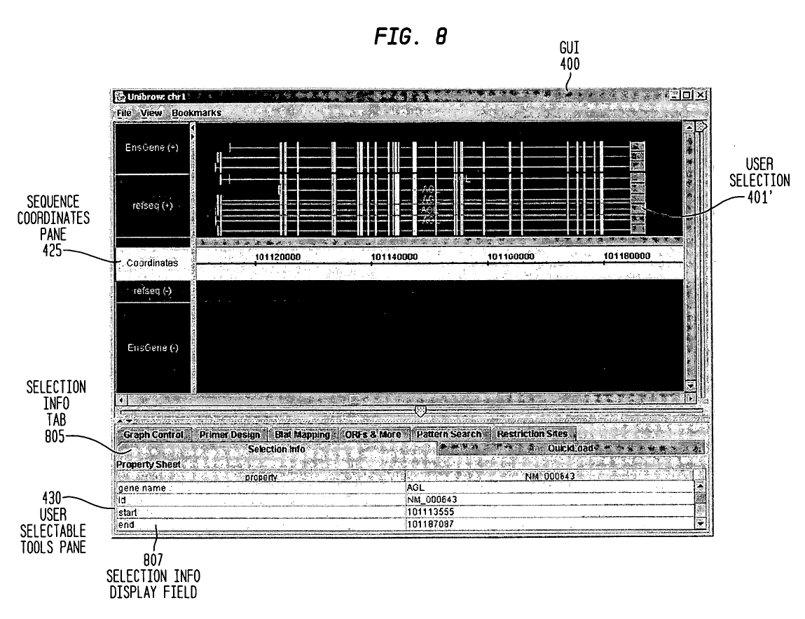

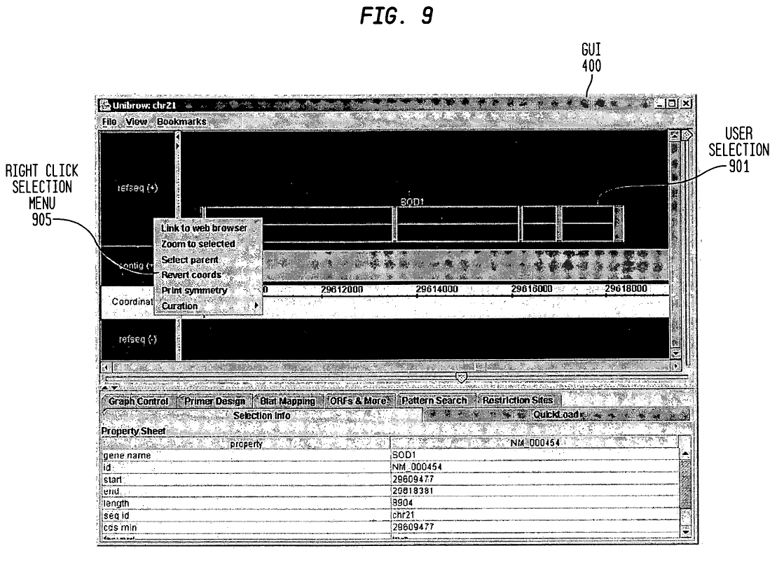

In the same or other implementations, the level of magnification along the horizontal axis of panes 405 and 407 may be controlled by other methods such as, for example, by a user selecting one or more elements displayed within panes 405, 407, and 425, illustrated in FIG. 4 as user selection 401. In the present example, the user may then select a magnification function from a menu, button, tab, or other methods of function selection commonly known to those of ordinary skill in the related art, such as, for instance, right click selection menu 905 illustrated in FIG. 9. Menu 905, in the present example, may be accessed by a user selection of the right button on a two button mouse. The display in menu 905 may include a “Zoom to selected” option that if selected by the user instructs GUI manager 211 to automatically increase the magnification to display the one or more user selections 401 such as illustrated in FIG. 8 as user selection 401′.

Additional dynamic features of the presently described implementation include vertical view selection bar 411 and horizontal view selection bar 413. Bars 411 and 413 may allow user 101 to interactively control what elements are displayed in panes 405 and 407. As previously described, as magnification increases either vertically or horizontally in panes 405 and 407, the amount of information displayed may be reduced and some information may be scrolled out of view off one or more vertical and/or horizontal edges. Bars 411 and 413 allow a user by methods commonly known to those of ordinary skill in the related art to select and control the information displayed in panes 405 and 407.

In some embodiments, an additional pane may be displayed that provides user 101 with a selection of tools that may be implemented by biological sequence tools 212, illustrated in FIG. 4 as user selectable tools pane 430. Tools pane 430 may provide a user with a plurality of selectable tools that could include one or more options that user 101 could apply to select information, display information and/or import information into generator 210, search biological sequence and/or related annotation information, produce results based upon analysis of sequence or related annotation information, or other tools known to those in the related art. In some embodiments, user 101 may select from the plurality of options using methods of selection commonly known to those in the art including selectable graphical elements such as tabs or buttons.

One tool that could be accessible by a user selectable tab is illustrated in FIG. 4 as quickload tab 432. In one possible embodiment user 101 may select tab 432 to instruct GUI manager 211 to display a plurality of additional user selectable options in pane 430. The plurality of user selectable options may include load sequence residues button 434 and annotated sequence selection button 436. For example, user 101 may desire to load a particular biological sequence and corresponding annotation information that may have been previously precompiled by the user or other source. In the present example, such information may be stored in biological sequence data 223 within executables 190A to maximize the speed and efficiency of loading large amounts of data and/or to provide a level of security for information the user may consider sensitive. User 101 may select button 436 that instructs GUI manager 211 to present a menu of one or more options of available sets of annotated sequence data 225 to load. The user could select one of the available sets of data by methods commonly known to those of ordinary skill in the related art. The selection instructs generator 210 to load the data, illustrated in FIG. 2 as annotated biological sequence data 225, from database 220.

Generator 210 may, in some embodiments, represent biological sequence data loaded from a remote source using data model 213. Alternatively, the biological sequence data could have been previously converted to the representation of data model 213 and saved by generator 210 in biological sequence data 223. GUI manager 211 may display one or more options in selected annotated sequence display field 438. The displayed options may include one or more sets of data within data 225 such as, for instance, the nucleotide sequence of a human chromosome. For example, user 101 may select one or more of the options displayed in field 438 by methods commonly known to those in the related art, for display in panes 405, 407, 420, and 425. Additionally in the present example a user may desire to load the biological sequence base representations or residues that correspond to sequence data 225. The user may select load sequence residues button 434 that instructs generator 210 to load the sequence residues, illustrated in FIG. 2 as sequence residues 229.

Some embodiments of generator 210 may be optimized for efficient loading and computing efficiency of data such as, for instance sequence residues that may be very computationally expensive to load in great numbers. For example, a possible method for efficient data loading may include a compressed representation of the data encapsulated in data model 213. For instance as those of ordinary skill in the related art will appreciate, instead of storing residues of a sequence as a string, they may alternatively be stored as an array of bytes where each residue may be represented as a 4-bit “nibble”. In the present example, the 4-bit nibble may also provide greater flexibility to generator 210 for working with data sets of variable size.

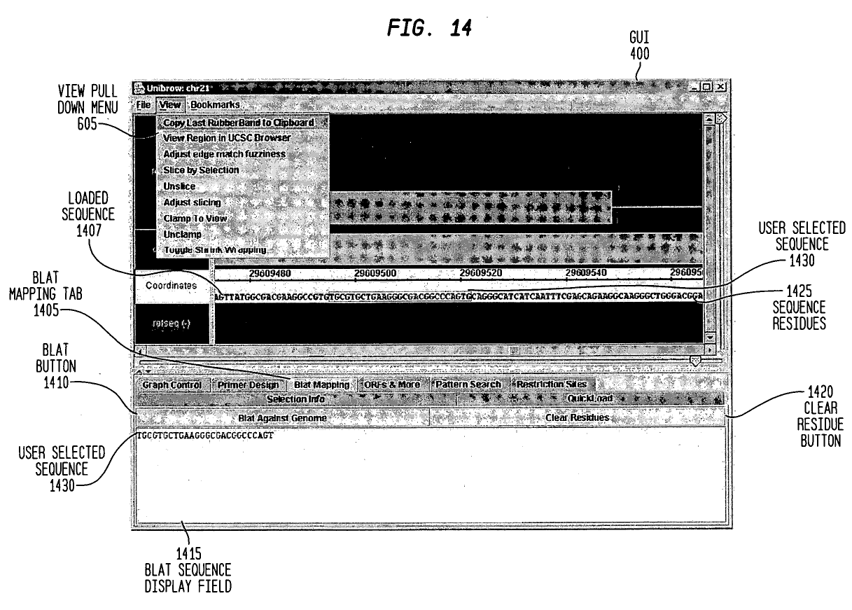

GUI manager 211 may display residues 229 in sequence coordinates pane 425 if the user selected magnification of selection bar 412 provides for a sufficiently fine resolution so that the individual bases may be displayed, such as is illustrated in FIGS. 14 through 17 as sequence residues 1425.

Another selectable tool that may be included in user selectable tools pane 430 may include an information selection tool accessible by a user selection of selection info tab 805. If user 101 selects an annotated gene in either of panes 405 or 407 such as, for instance, user selection 401′ as illustrated in FIG. 8, one or more fields of descriptive information corresponding to that gene may be displayed in selection info display field 807. For example, the one or more fields of descriptive information may include gene name, gene identifier, start position coordinates of the gene counted from one end of the loaded sequence, end position coordinates of the gene counted from the same position as the start position coordinates, length of the gene in number of base residues, sequence identifier, coding sequence start position, DNA strand (i.e. plus, minus, forward, reverse, coding, non-coding), annotation source, coding sequence stop coordinates, or other related information.

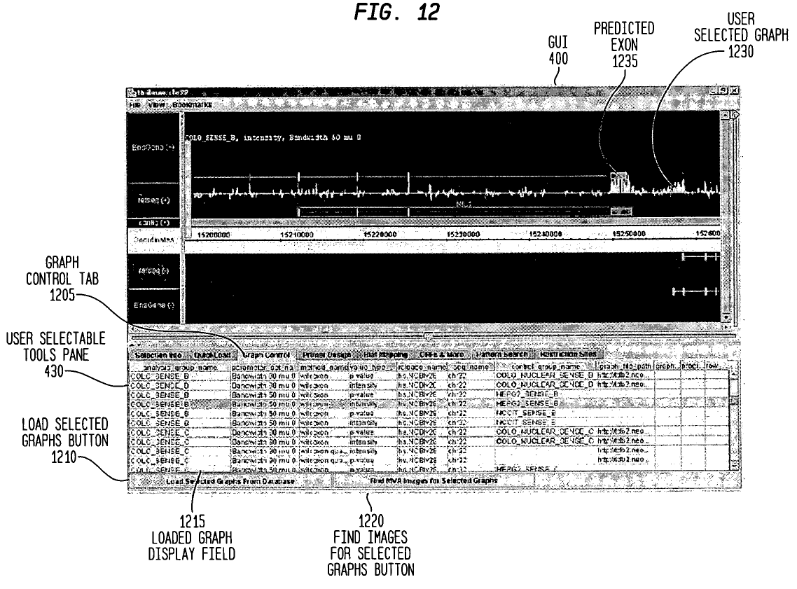

FIG. 12 provides an illustrative example of another tool that may, in some implementation, be included in user selectable tools pane 430. The tool may be accessible by a user selection of graph control tab 1205. Once selected one or more additional buttons for user selections may be displayed that could include load selected graphs button 1210 and find images for selected graphs button 1220. In some embodiments button 1220 may allow user 101 to search for precompiled graphs from a remote source or local source such as for instance from database 220, additionally, button 1210 may allow a user to load sequences. In some implementations, when user 101 selects load selected graphs button 1210, generator 210 loads one or more graphs from database 220, illustrated in FIG. 2 as precompiled graphs 227. GUI manager 211 may then display graphs 227 in loaded graph display field 1215 that may, for instance, include one or more user selectable options of graphs to display. Display field 1215 may additionally include a plurality of sub fields that displays descriptive information corresponding to each available graph. One example of a graph is presented in FIG. 12 as user selected graph 1230. In the present example the graph may represent experimental results from one or more experiments that could, for instance, include experiments relating to what is referred to as the transcriptome.

Graph 1230 may include one or more graphical elements such as colored bars where the height of each of the graphical bar elements may reflect the relative abundance of a transcript that may, for instance, be associated with the hybridization of biological transcripts to probes disposed upon biological probe arrays such as hybridized probe arrays 103. For example, at fine resolutions each bar may represent the detected emission intensity from a single probe. Additionally, the graphs could provide a means for interpretation of experimental results. For instance, as illustrated in FIG. 12 graph 1230 displays a region that has a high level of detected transcript abundance, illustrated by the height of the bar elements that correspond to predicted exon 1235. Alternatively, other regions of the graph show high levels detected transcript abundance that have no predicted exon regions that correspond to them. Such regions may represent previously unknown exons or genes, regulatory elements, or other interesting features.

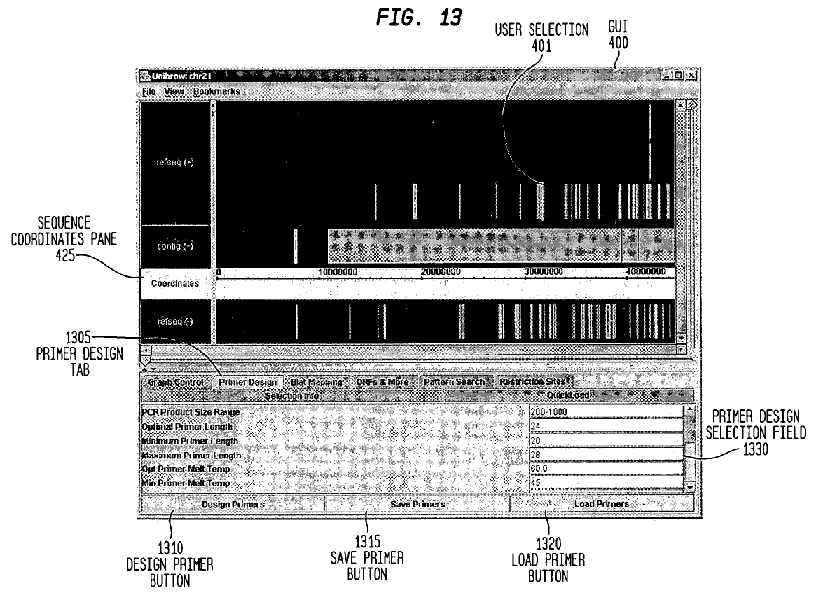

FIG. 13 provides yet another example of a user selectable tool provided in pane 430 illustrated as primer design tab 1305. The tool accessed by tab 1305 may provide user 101 with a fast and efficient method for what is referred to by those of ordinary skill in the related art as primer design. Primers are commonly used with an experimental technique referred to as polymerase chain reaction or PCR. For instance, in some uses of the PCR technique the primers define what are referred to as the 3′ and 5′ ends of a region of nucleotide sequence that a user may wish to create many copies of.

Selection of tab 1305 initiates a display of a plurality of selectable buttons that provides user 101 access to features provided by the tool. Additionally, one or more default primer design options may be displayed in primer design selection field 1330 that could, for example, include one or more parameters commonly used by those of ordinary skill in the related art for primer design. In the present example, user 101 may change any of the default options to a different value. In the present example, the primer design options may include PCR product size range, optimal primer length, minimum primer length, maximum primer length, optimal primer melting temperature, minimum primer melting temperature, maximum primer melting temperature, minimum primer % GC content, maximum primer % GC content, salt concentration, DNA concentration, Maximum number of unknown bases, maximum self-comp, maximum 3′ self comp, and GC clamp.

The selectable buttons of the primer design tool may include design primer button 1310, save primer button 1315, and load primer button 1320. In some implementations, the sequence residues may be loaded into generator 210 by methods previously outlined prior selection of tab 1305. For example, when user 101 selects design primer button 1310, generator 210 may use one or more of the design options listed above as parameters to design what is referred to as a primer set for one or more sequences identified by user selection 401. In the present example, generator 210 may present the designed primer set to user 101 in primer design selection field 1330 and/or as a sequence aligned to the displayed sequence in sequence coordinates pane 425.

Illustrated in FIG. 14 is an additional tool accessible via selection of BLAT mapping tab 1405 in pane 430 for what is referred to as the Basic Local Alignment Tool or BLAT. BLAT includes an alignment tool similar to the well known BLAST alignment and search tool, but is structured differently such as, for example, by keeping an index of the entire genome searched in memory. Thus the BLAT tool is faster than BLAST and performs well with both nucleic acid sequences as well as protein sequence. Also, in the case of nucleic acid sequences may find sequences of 95% or greater similarity from queries of a length of 40 bases or more. Some implementations of biological sequence tools 212 use the BLAT algorithm as a tool that aligns a user input or selected query sequence with one or more sequences loaded into generator 210 such as loaded sequence 1407. User 101 may select the query sequence from a local or remote source and type or paste by commonly used methods into BLAT sequence display field 1415. Selection of BLAT button 1410 instructs GUI manager 211 to align the query sequence pasted into display field 1410 to loaded sequence 1407 using the BLAT algorithm.

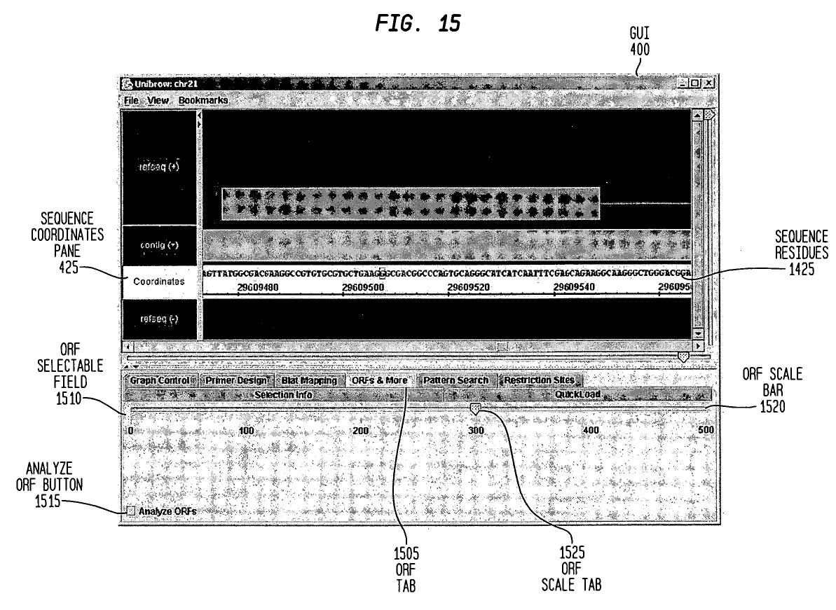

Some embodiments of biological sequence tools 212 may include another tool of pane 430 that may be available for analyzing a loaded or user selected sequence region for what is commonly referred to as an open reading or translation frame. Typically, for what are referred to as eukaryotes, three nucleotide bases typically code for each translated protein base. The three nucleotide bases are commonly referred to as a codon that may be read by a cell's translation machinery in what is commonly referred to as the translation or reading frame. Each sequence of DNA has six possible reading frames, three in each direction. Typically, only one reading frame codes for a protein and is referred to as the open reading frame. As is known to those of ordinary skill in the related art, the open reading frame typically begins with what is referred to as a start codon, and ends with a stop codon. The open reading frame analysis tool may be accessible by a user selection of ORF tab 1505 as illustrated in FIG. 15. Upon selection of tab 1505 ORF scale bar 1520 may be displayed in ORF selectable field 1510. In some implementations, the scale bar may represent a selectable minimum size of the ORF to be identified in loaded sequence 1407 or selected sequence such as, for instance, selected sequence 1430 of FIG. 14. User 101 may interactively select a value represented on scale bar 1520 by moving ORF scale tab 1525, via commonly used methods such as clicking and dragging with a mouse, to the desired position along scale bar 1520. In the illustrated implementation, scale bar 1520 may use a variety of different incremental scales, such as for instance numbers of base residues, as well as what is referred to by those of ordinary skill in the related art as kilobases, megabases, centimorgans, or other incremental value used for sequence measurement. In some embodiments, tab 1525 may be set to some default value that could correspond to an average ORF size or some other value. A selection of analyze ORF button 1515 instructs generator 210 to find one or more open reading frames in a loaded sequence or user selection of sequence, using the user selected criteria of scale tab 1525. GUI manager 211 may return the results to the user in a variety of formats that could include one more colored boxes displayed in sequence coordinates pane 425 aligned with the one or more identified ORF's of sequence residues 1425.

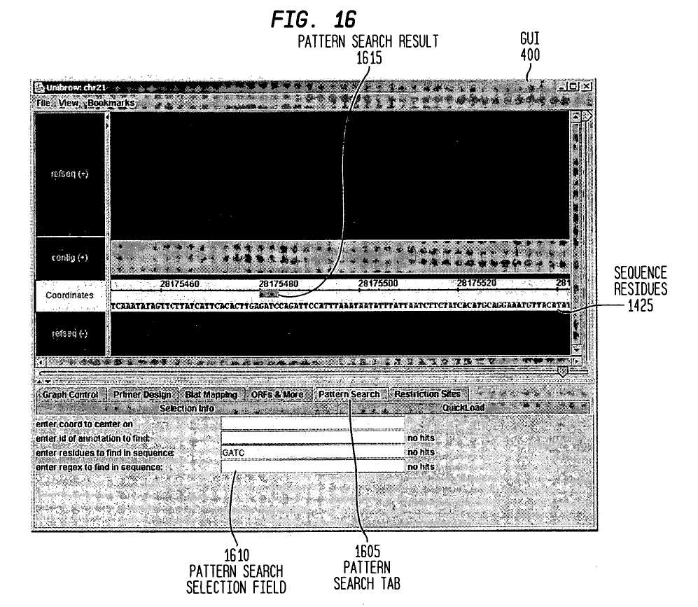

Yet another tool of pane 430 could include a pattern search tool accessible by a user selection of pattern search tab 1605. The pattern search tool may perform a variety of searches for information within a loaded sequence that, for example, could include searching for a gene or annotation by a user input identifier, searches for perfect matches to user input sequence, what is referred to as regular expression matching that can define variable parameters for sequence matching, centering search parameters on specific coordinates, or other type of search useful for mining information out biological sequence data. In the present example, a user may type or paste a sequence into one or more fields within pattern search selection field 1610 such as, for instance, for a perfect match search. Biological sequence tools 212 finds all perfect matches to the user input sequence within a loaded sequence, such as is illustrated in FIG. 16 as sequence residues 1425. Generator 210 may display the result as a color coded bar such as illustrated in FIG. 16 as pattern search result 1615. It will be appreciated by those of ordinary skill in the related art that the previous example is for purpose of illustration and should not be limiting in any way.

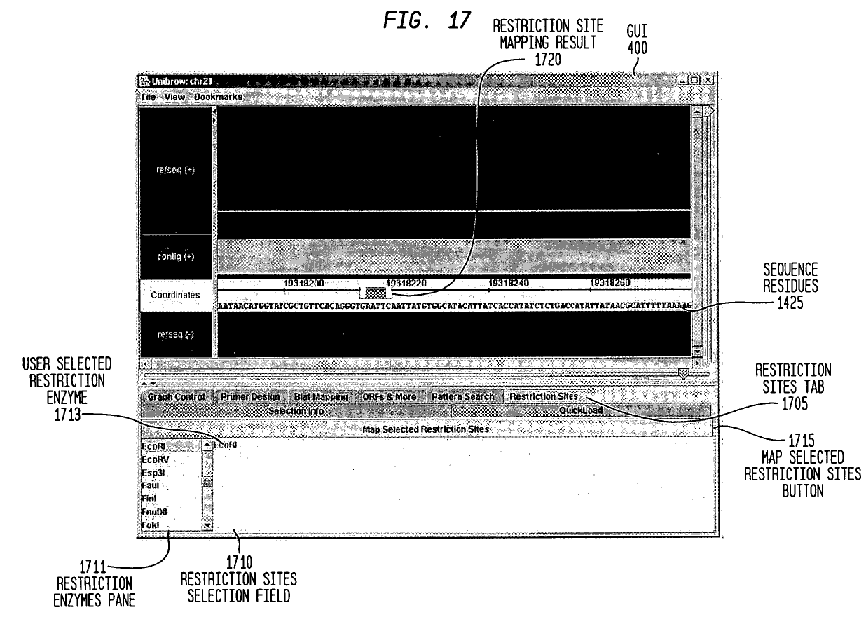

Illustrated in FIG. 17 is another possible tool accessible in pane 430 for mapping what is commonly referred to as restriction sites to a loaded sequence. As will be appreciated by those of ordinary skill in the related art, restriction sites typically refer to specific sequences targeted by what are referred to as restriction enzymes to cleave or cut DNA. Selection of restriction sites tab 1705 instructs GUI manager 211 to display one or more panes within restriction sites selection field 1710. The one or more panes may include restriction enzymes pane 1711 that may display a plurality of known restriction enzymes that additionally may be selectable by user 101. For example, user 101 may select one or more restriction enzymes whose known target sequence may be mapped to all instances of the corresponding sequence within a loaded or user selected sequence. In the present example, user 101 may select a restriction enzyme, such as user selected restriction enzyme 1713 that for instance may include EcoRI. The user may then select map restriction sites button 1715 that instructs sequence tools 212 to find and identify all of the target sites of the EcoRI enzyme within sequence residues 1425. GUI manager 211 returns the results in one or more panes of GUI 400 that may include restriction site mapping result 1720. Result 1720 as illustrated in FIG. 17, may include a colored box that corresponds to the identified target sequence within residues 1425.

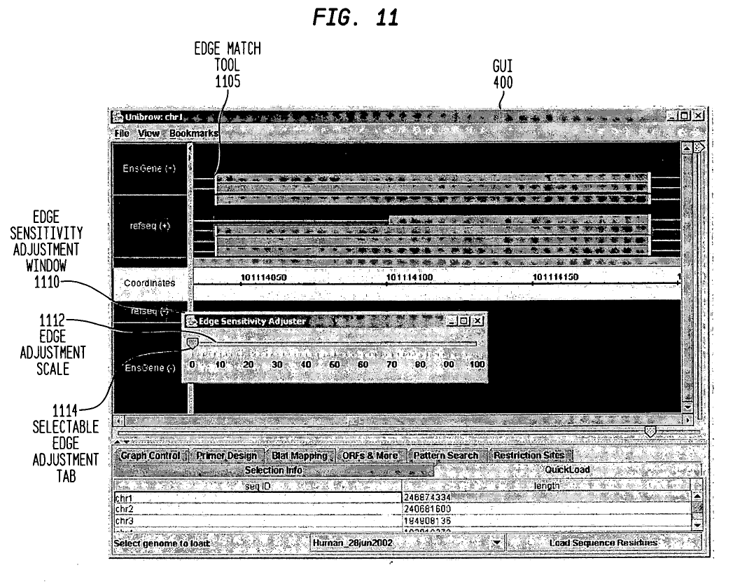

Additionally, biological sequence tools 212 may include other tools accessible via means other than through pane 430. One such tool may include what will hereafter be referred to as the edge match tool. The edge match tool is illustrated in FIG. 11 as edge match tool 1105. In some implementations, edge match tool 1105 may be automatically activated upon a user selection of one or more sequence annotations 403, regions of sequence contig 404, sequence residues 1425, or other type of sequence related selection. Illustrated in FIG. 4 is sequence selection 401 that for instance may be graphically highlighted by a color or pattern change. Selection 401 is further illustrated in FIG. 8 at a higher magnification displaying highlighted regions of the “edges” of annotated exons that are aligned together. In the presently described implementation, edge match tool 1105 may include edge sensitivity adjustment window 1110, that could for instance be accessible in view pull down menu 605. Edge sensitivity adjustment window 1110 provides a means for a user to interactively select what may be referred to as the edge “fuzziness” of the aligned edges. Adjustment window 1110 may include a scale such as edge adjustment scale 1112 that may provide increments that user 101 may interactively select. Adjustment of scale 1112 may change one or more parameters that define what tools 212 considers an edge. For example, a default setting may include an edge fuzziness of 0 bases that means the alignment of the edges of the annotated exons must be perfectly aligned. Alternatively, if a user selects and moves selectable edge adjustment tab 1114 to another value that could, for instance, include a value of 50 bases, then tools 212 defines a plurality of edges as matched if the exon boundaries are within 50 bases of the edge of the exon defined by user selection 401.

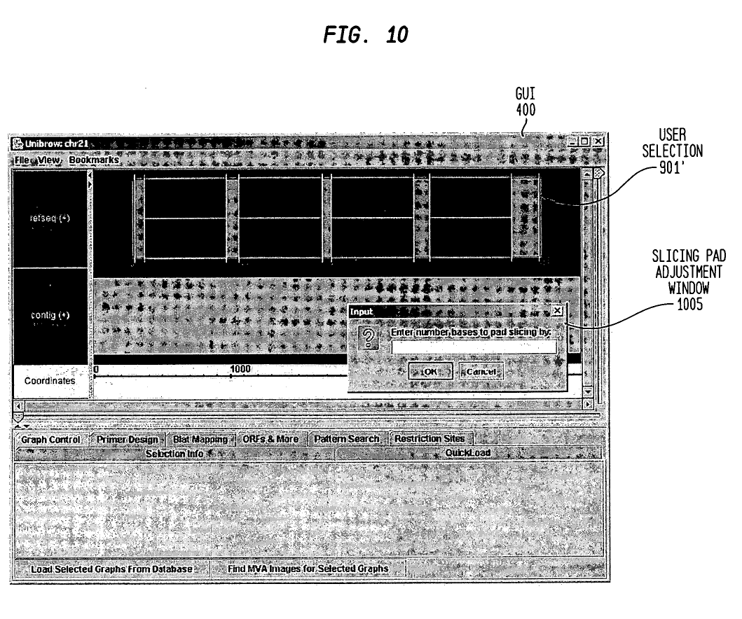

In some embodiments, biological sequence tools 212 may additionally provide a tool referred to as the slice by selection tool. The slice by selection tool may be accessed by a variety of methods that could include a selectable option in view pull down menu 605. The slice by selection tool may change how a user selection, such as user selection 401, is displayed in panes 405 and 407. The slice by selection tool may “pad” into the introns by defined number of bases that splice exons together. The defined number of bases that tools 212 uses to pad into the introns may be a default value that could for instance be optimized for most gene annotations, or a user selectable value. Another selectable option that may be available in view pull down menu 605 is an “adjust slicing” option. Upon selection of the “adjust slicing” option, GUI manager 211 may display an additional window that could, for instance, include slicing pad adjustment window 1005. Window 1005 may provide user 101 one or more fields to type or paste a value for the number of bases for tools 212 to use as a parameter. For example, illustrated in FIG. 9 is user selection 901 that displays a predicted annotated gene from the RefSeq database. Illustrated in FIG. 10 is user selection 901′ that GUI manager 211 has displayed after a user selection of the slice by selection tool. Selections 901 and 901′ illustrate how the same annotation may be viewed so that the exon structure may be more clearly viewed. Additionally, the scale in sequence coordinates pane 425 may reflect the length of user selection 901′ rather than the position within the loaded sequence as illustrated in FIG. 9 with respect to user selection 901.

In some embodiments a tool may be provided for what those in the related art refer to as curation or hand curation of biological sequence and sequence related information. The curation tool may be accessible by a variety means including, for instance curation menu 1805. The curation tool may additionally provide the means to save curations, load saved curations, and edit or manipulate curations. For example, if a user disagrees with the annotated gene prediction for a given region of biological sequence, the user may interactively select sequence regions, predicted exons, or other elements displayed in panes 405 and 407, as a curation that the user may believe to be more accurate.

Tools 212 may also provide additional tools in a plurality of menus that could include file pull down menu 505, view pull down menu 605, bookmark pull down menu 705, right click selection menu 905, and curation menu 1805. For instance, bookmark pull down menu 705 may allow a user to save information relating to the loaded biological sequence as a “bookmark”. Such information could include sequence contig 404, sequence annotations 403, one or more user selections 401, or other related information. Additionally, a user may export or import bookmarks to and from local and or remote workstations or servers.

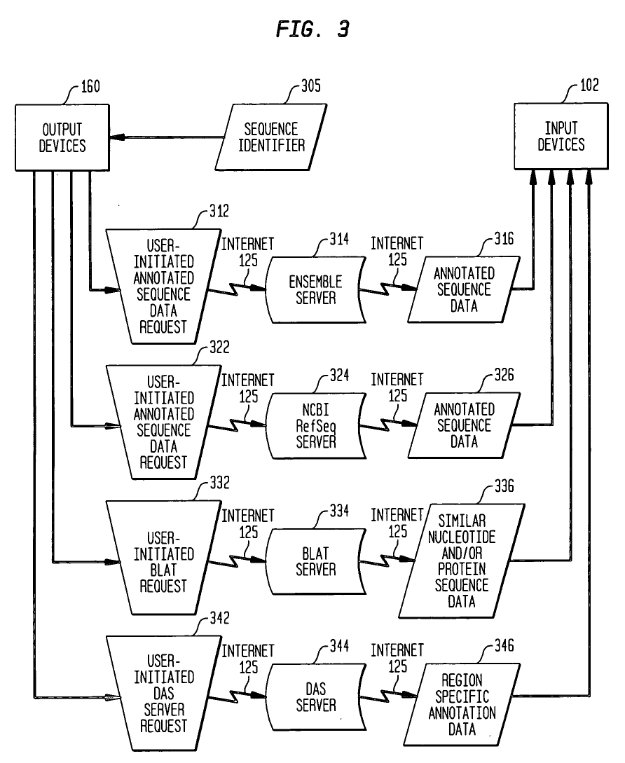

As illustrated in FIG. 3, some tools may use links to local and/or remote workstations or database servers. For example, internet 125 could be used to access information provided by a remoter database server such as ensembl server 314, NCBI RefSeq server 324, BLAT server 334, and DAS server 344.

Generator 210 may link directly to one or more of the remote data severs. Alternatively generator 210 may use what is referred to by those of ordinary skill in the related art as a servlet to link to remote data sources, illustrated in FIG. 2 as dynamic display servlet 226. In some embodiments a servlet may provide an open line of communication between generator 210 and one or more remote data sources such as BLAT server 334 and/or DAS server 344.

In some embodiments tools 212 may query servers 314 or 324 based on user-initiated annotated sequence data request 312 or 322 that could include one or more user selections of, for example, sequence annotations such as user selection 401. In the presently described embodiments selection 401 may identify one or more sequence identifiers 305 that may be used to directly query servers 314 or 324. Servers 314 and 324 may return corresponding information, illustrated in FIG. 3 as annotated sequence data 316 and 326, by a variety of methods including opening a window of a web browser to display annotated sequence data 316 or 326.

In the same or other embodiments, generator 210 may employ servlet 226 for communication with one or more remote data sources, such as servers 334 and 344. Servlet 226 may be implemented as a Java servlet, CGI program, or other type of implementation. Servlet 226 may respond to user-initiated BLAT request 332 as previously described in reference to a user selection of BLAT mapping tab 1405. Additionally, servlet 226 may respond to user-initiated DAS server request 342 that for instance could include a selection from file pull down menu 505 that may provide a user with DAS window 1850. For example, a plurality of fields may be displayed in DAS window 1850 that may include one or more pull down menus. The one or more pull down menus may provide the user with selectable options for available DAS servers or other data sources. In the present example when the user selects a DAS server, such as for instance DAS server 344, information may be displayed in the plurality of fields displayed in window 1850 that corresponds to the sequence information displayed in panes 405 and 407, such as contig 404. The displayed information may include a sequence identifier, a minimum range, and a maximum range. Additionally in the present example, servlet 226 may provide a connection that could allow DAS server 344 to export data, such as region specific annotation data 346, directly into generator 210. GUI manager 211 may then display the data received from DAS server 344 in one or more panes of GUI 400 such as panes 405 and/or 407. Another example of a Distributed Annotation Server is provided in U.S. Provisional Application Serial No. 60/444,952, titled “DAS2: A Distributed Genome Annotation System”, filed Feb. 3, 2003, which is hereby incorporated by reference in its entirety for all purposes.

Servlet 226 may also provide additional functionality such as maintaining an open connection via internet 125 that could allow one or more remote sources to access generator 210 without a query from generator 210. For example, a user may make a selection of a probe set or other gene or sequence identifier in a web browser interface. The remote portal linked to the web browser interface may then utilize the open connection to generator 210 and export data corresponding to the user selection into generator 210. In the present example of a user selection of a probe set, graphical elements depicting the probe set could be displayed in panes 405 and/or 407, as well as probe sequences displayed in coordinates pane 425, or displays of other related information.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways and by various elements in alternative embodiments. For example, some or all of the functions described as being carried out by dynamic display application 190 could be carried out by probe-array analysis applications 199 or these functions could otherwise be distributed among other functional elements. Also, the functions of several elements may, in alternative embodiments, be carried out by fewer, or a single, element. For example, the functions of dynamic display application 190 and probe-array analysis applications 199 could be carried out by a single element in other implementations. Similarly, in some embodiments, any functional element may perform fewer, or different, operations than those described with respect to the illustrated embodiment. Also, functional elements shown as distinct for purposes of illustration may be incorporated within other functional elements in a particular implementation. For example, the division of functions between an application server and a network server of the genome portal is illustrative only. The functions performed by the two servers could be performed by a single server or other computing platform, distributed over more than two computer platforms, or other otherwise distributed in accordance with various known computing techniques.

Also, the sequencing of functions or portions of functions generally may be altered. Certain functional elements, files, data structures, and so on, may be described in the illustrated embodiments as located in system memory of a particular computer. In other embodiments, however, they may be located on, or distributed across, computer systems or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel or distributed processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

What is claimed is:

1. A method for representing biological sequences, comprising the acts of:
receiving a plurality of related biological sequences; and
representing each of the plurality of related biological sequences as a plurality of objects of a data model, wherein at least one of the plurality objects is enabled to map at least one of the plurality of related biological sequences with at least one other of the plurality of related biological sequences.

2. The method of claim 1, wherein:
the plurality of related biological sequences include, protein, mRNA, genomic, and probe sequences.

3. The method of claim 1, wherein:
the plurality of objects includes a BioSeq object, wherein the BioSeq object represents the length and sequence composition of a subsequence of one of the plurality of related biological sequences.

4. The method of claim 3, wherein:
the plurality of objects includes a SeqSpan object, wherein the SeqSpan object represents the start and end positions of the subsequence and points to the BioSeq object.

5. The method of claim 4, wherein:
the plurality of objects includes a SeqSymmetry object, wherein the SeqSymmetry object represents and points to one or more of the SeqSpan objects.

6. The method of claim 5, wherein:
the plurality of objects includes an AnnotatedBioseq object, wherein the AnnotatedBioseq object represents and points to the SeqSymmetry object and one or more SeqSpan objects associated with at least one other of the plurality of related biological sequences.

7. A system for representing biological sequences, comprising:
an input manager constructed and arranged to receive a plurality of related biological sequences; and
a dynamic display generator constructed and arranged to represent each of the plurality of related biological sequences as a plurality of objects of a data model, wherein at least one of the plurality objects is enabled to map at least one of the plurality of related biological sequences with at least one other of the plurality of related biological sequences.

8. The method of claim 7, wherein:
the plurality of related biological sequences include, protein, mRNA, genomic, and probe sequences.

9. The method of claim 7, wherein:
the plurality of objects includes a BioSeq object, wherein the BioSeq object represents the length and sequence composition of a subsequence of one of the plurality of related biological sequences.

10. The method of claim 9, wherein:
the plurality of objects includes a SeqSpan object, wherein the SeqSpan object represents the start and end positions of the subsequence and points to the BioSeq object.

11. The method of claim 10, wherein:
the plurality of objects includes a SeqSymmetry object, wherein the SeqSymmetry object represents and points to one or more of the SeqSpan objects.

12. The method of claim 11, wherein:
the plurality of objects includes an AnnotatedBioseq object, wherein the AnnotatedBioseq object represents and points to the SeqSymmetry object and one or more SeqSpan objects associated with at least one other of the plurality of related biological sequences.
